(19) United States
(12) Patent Application Publication
(10) Pub. No.: US 2013/0121879 A1
(43) Pub. Date: May 16, 2013

(54) Magnetic-Field Measurement Device

(71) Applicants: Ryuzo Kawabata, Higashiyamato (JP); Takako Mizoguchi, Sayama (JP); Akira Tsukamoto, Toda (JP); Akihiko Kandori, Tokyo (JP); Tomoko Yoshimura, Ichikawa (JP)

(72) Inventors: Ryuzo Kawabata, Higashiyamato (JP); Takako Mizoguchi, Sayama (JP); Akira Tsukamoto, Toda (JP); Akihiko Kandori, Tokyo (JP); Tomoko Yoshimura, Ichikawa (JP)

(73) Assignee: Hitachi, Ltd., Chiyoda-ku, Tokyo (JP)

(21) Appl. No.: 13/810,832

(22) PCT Filed: Jul. 19, 2011
(86) PCT No.: PCT/JP2011/066389

§ 371 Date: Jan. 17, 2013

(30) Foreign Application Priority Data
Jul. 21, 2010 (JP) 2010-163997

(51) Int. Cl. G01N 33/543

(52) U.S. Cl. CPC G01N 33/54333
USPC 422/69

(57) ABSTRACT

The disclosed magnetic immunoassay device, which performs magnetic immunoassays using antigen-antibody reactions, can perform speedy immunoassays without bound/free separation in the test samples. The device is also practical, being capable of stable magnetism measurement without magnetic shielding. The disclosed magnetic immunoassay device is provided with: an excitation coil that uses an AC magnetic field to magnetize a test sample containing a magnetic marker; a magnetism sensor that measures magnetism in the test sample and outputs a magnetism signal; and a displacement sensor for detecting changes in the distance between the test sample and the magnetism sensor. By optimally setting the bandwidth of a lock-in amplifier, which detects changes in the phase of the magnetism signal outputted by the magnetism sensor, and the rotational speed produced by a drive system, which moves the test sample at low speeds, the impact of environment magnetic noise is reduced, and correcting the magnetism signal using distance information obtained from the displacement sensor allows stable magnetism measurement.

### TECHNICAL FIELD

The present invention relates to a magnetic-field measurement device, for example, relates to an immunoassay technology which applies an AC magnetic field to a measurement sample that includes a small magnetic particle and detects an antigen-antibody reaction by a magnetic method.

### BACKGROUND ART

An immunoreaction is widely used in various fields from a detection of a germ or a cancer cell in foods over to a detection of an environmental harmful substance causing allergy or the like. The immunoreaction is caused by coupling a measurement object substance (antigen) and an inspection reagent (antibody) which selectively couples with the antigen, and a kind and an amount of the antigen are measured from the coupling. In an inspection using such an immunoreaction, a marker is added to the antibody for using a reaction of coupling an antigen and antibody (antigen-antibody reaction). An optical marker is generally used for the marker, and the antigen-antibody reaction is detected by an optical measurement.

In recent years, despite high needs for detecting an extremely small antigen-antibody reaction with a high sensitivity at a high speed, there is brought about a limit since a solid phase method is used as a cleaning step that is referred to as BF (Bound/Free) separation. According to the solid phase method, when an antibody marked by a marker (detection antibody) is put into an inspection vessel into which a board added with an antibody (fixed antibody) is put, a portion of the marker is brought into a coupling state (coupling marker) which interposes an antigen by the fixed antibody and the detection antibody, and the remaining marker stays to be an uncoupling state (uncoupling marker). When the uncoupling marker is present in the inspection vessel, the coupled antibody cannot be identified by the immunoassay by the optical measurement. Therefore, it is necessary to wash away the uncoupled marker in the inspection vessel by BF separation. The BF separation takes time and labor, and therefore, the BF separation becomes a significant factor of hampering speedy inspection.

On the other hand, there is carried out a new method (magnetic immunoassay) which magnetically detects an antigen-antibody reaction by using a magnetic small particle (hereinafter, referred to as magnetic marker) as an immunoassay which does not have BF separation (refer to Nonpatent Literatures 1-9). It is also reported that the magnetic immunoassay achieves an immunoassay with a sensitivity 10 times or more as high as that of the optical method of the background art by using a superconductor SQUID (Superconducting Quantum Interference Device) fluxmeter for a magnetism sensor.

In the magnetic immunoassay, there are measurement methods by (1) susceptibility measurement, (2) magnetic relaxation measurement, and (3) residual magnetism measurement. An explanation will be given of respective measurement methods as follows.

### (1) Concerning Susceptibility Measurement

When an inspection sample into which a magnetic marker magnetized by a DC magnetic field is put passes through a superconductor SQUID fluxmeter, a magnetism signal from the inspection sample is detected by the superconductor SQUID fluxmeter (refer to, for example, Patent Literature 1, Nonpatent Literature 1, Nonpatent Literature 2, and Nonpatent Literature 3). At that occasion, a direction of applying the DC magnetic field and a detecting direction of the superconductor SQUID fluxmeter are arranged to be orthogonal to each other. There is also a case of using an AC magnetic field for magnetizing an inspection sample (refer to, for example, Patent Literature 2, Nonpatent Literature 4).

### (2) Concerning Magnetic Relaxation Measurement

An inspection sample into which a magnetic marker is put is fixed to a detecting position of a superconductor SQUID fluxmeter, and a pulse magnetic field of 1 mT is applied to the inspection sample. At this occasion, a direction of applying the DC magnetic field and the detecting direction of the superconductor SQUID fluxmeter are arranged to be orthogonal to each other. The superconductor SQUID fluxmeter detects a relaxation of a magnetism signal from the sample for 1 second immediately after applying the pulse magnetic field. The magnetic marker is magnetized by applying the pulse magnetic field, and a residual magnetism is generated at the magnetic marker immediately after applying the magnetic field. The residual magnetism is reduced over time by a thermal noise. In the magnetic relaxation measurement, an immunoassay is carried out by the relaxation of the residual magnetism from the coupling marker by making use of a difference between the relaxation time periods of a magnetic marker coupled to an antigen in the sample (coupling marker) and a magnetic marker which is not coupled to the antigen (uncoupling marker) (refer to, for example, Nonpatent Literature 1, Nonpatent Literature 5, Nonpatent Literature 6, and Nonpatent Literature 7).

(3) Residual Magnetism Measurement When a size of the magnetic marker is increased, a residual magnetism is not relaxed in a case where the magnetism marker is magnetized. In the residual magnetism measurement, a residual magnetism is generated at the magnetic marker by applying a magnetic field of about 0.1 T to an inspection sample into which the magnetic marker is put at a position remote from the superconductor SQUID fluxmeter. Thereafter, an inspection vessel into which the inspection sample is put is moved, and the residual magnetism is detected by the superconductor SQUID fluxmeter (refer to, for example, Nonpatent Literature 1, Nonpatent Literature 8, Nonpatent literature 9).

### CITATION LIST

### Patent Literature

Patent Literature 1: Japanese Unexamined Patent Application Publication No. 2001-33455
Patent Literature 2: Japanese Unexamined Patent Application Publication No. 2001-133458

### Nonpatent Literature

Nonpatent Literature 1: Keiji Enpuku: Japan Journal of Applied Physics Society Vol. 73, No. 1, p. 28 (2004)
Nonpatent Literature 2: K. Enpuku et al.: IEEE Trans Appl. Supercond. 11, p. 661 (2001)
Nonpatent Literature 3: K. Enpuku et al.: Jpn. J. Appl. Phys. 38, p.L1102 (1999)
Nonpatent Literature 4: S. Tanaka et al.: IEEE Trans. Appl. Supercond. 11, p. 665 (2001)
Nonpatent Literature 5: Y. R. Chemla et al.: Proc. Natl. Acad. Sci. USA 97, p. 14268 (2000)
Nonpatent Literature 6: A. Haller et al.: IEEE Trans. Appl. Supercond. 11, p. 1371 (2001)
Nonpatent Literature 7: S. K. Lee et al. Appl. Phys. Lett. 81, 3094 (2002)
Nonpatent Literature 8: R. Kotitz et al.: IEEE Trans. Appl. Supercond. 7, p. 3678 (1997)
Nonpatent Literature 9: K. Enpuku et al.: Jpn. J. Appl. Phys. 42, p.L1436 (2003)

### SUMMARY OF INVENTION

### Technical Problem

According to a magnetic immunoassay apparatus of a background art, there is used the superconductor SQUID fluxmeter which needs a coolant system (liquid nitrogen) and a vacuum system (vacuum pump) in the magnetism sensor, and therefore, there poses a serious problem in reduction of a clinical inspection apparatus to practice in view of large-sized formation of the apparatus and cost. Heretofore, according to the magnetic immunoassay, attention is paid to detecting an antigen-antibody reaction by a small amount of a magnetic marker with a high sensitivity, and there is not carried out a specific proposal of reducing the magnetic immunoassay into practice as an inspection system.

According to the magnetic immunoassay apparatus of the background art, an inspection sample into which a magnetic marker is put is magnetized, a magnetism signal from the magnetized inspection sample is detected, and therefore, it is necessary to cover the superconductor SQUID fluxmeter which is a sensor unit or a total of the apparatus by magnetic shielding. Although the magnetic shielding is effective for reducing an environmental magnetic noise entering into the magnetism sensor, the magnetic shielding is very expensive particularly in the size of covering the total of the apparatus since a material of the magnetic shielding consists of a rare metal. Also, a magnetic shielding property of the magnetic shielding is changed by a mechanical impact, and therefore, caution is required for handling the magnetic shielding.

According to the magnetic immunoassay apparatus of the background art, in a case of carrying out the measurement by moving the inspection sample, a magnetism caused by a moving device (for example, drive motor or the like) effects an influence on the measurement as a magnetic noise. Therefore, there is taken a countermeasure thereagainst of using a supersonic motor which does not generate magnetism in the moving device. The supersonic motor has an excellent characteristic of not emitting a magnetic noise since a magnetic material is not used at the driving unit different from a general motor. However, the supersonic motor is not only very expensive in comparison with the general motor, but an operating condition (continuous driving only for a short time period and short life) is delicate.

An apparatus configuration resolving the above-described problem is indispensable from a view point of cost and quality control in order to reduce the magnetic immunoassay apparatus to practice.

Hence, it is an object of the present invention to provide an immunoassay technology which realizes a highly sensitive and stable operation without magnetic shielding.

### Solution to Problem

The present invention has the following apparatus configuration as shown in FIG. 1 in order to realize the above-described object.

The magnetic immunoassay apparatus according to the present invention includes an excitation coil 101 for magnetizing an inspection sample into which a magnetic marker is put and an AC signal generator 107 which becomes a signal source of the excitation coil 101, and generates AC magnetism from the excitation coil 101. The inspection sample is installed on a circumference of a non-magnetic plate 103 of a circular disk type. The non-magnetic plate is rotationally moved by a drive unit configured by a DC motor 105. Incidentally, a motor driver 110 of the drive unit has a function of adjusting a rotational speed to be able to freely change a rotational speed of the non-magnetic plate 103.

The magnetic immunoassay apparatus of the present invention includes a magnetoresistance effect element (MR sensor), and detects a magnetism signal from the inspection sample that is magnetized by an AC magnetism from the excitation coil 101 by the MR sensor 104 when the non-magnetic plate 103 passes through a vicinity of the excitation coil 101 by the drive unit. The MR sensor 104 includes a small-sized coil for generating a signal of canceling the AC magnetism entering the MR sensor 104. It is necessary to synchronize a canceling magnetism signal and an excitation magnetism signal, and therefore, a signal source of the small-sized coil is made to be the AC signal generator 107 described above. An output of the AC signal generator 107 is inputted to the small-sized coil via an amplitude-phase adjustor 108 which adjusts intensity and a phase of a signal.

The magnetic immunoassay apparatus of the present invention includes a lock-in amplifier 109, an output of the MR sensor 104 and an output of a signal source of the excitation coil 101 are respectively made to be an input signal and a reference signal to the lock-in amplifier 109, and a change in a phase of the magnetism signal from the inspection sample magnetized by the excitation coil 101 is detected by the lock-in amplifier 109. The magnetic immunoassay apparatus also includes an A/D converter 112 for A/D-converting an output of the lock-in amplifier 109, and includes a data collector 113 for collecting a signal outputted from the A/D converter 112. The lock-in amplifier 109 has a function of capable of adjusting the magnetism signal from the inspection sample to an optimum detection band in accordance with a rotational speed of the non-magnetic plate 103 described above.

When the immunoassay apparatus, the magnetism signal detected from the inspection sample becomes very small depending on a concentration of the antigen which becomes an inspection object or an amount of the magnetic marker used. Therefore, there is a case in which a detecting signal waveform in real time is not clear. Hence, the magnetic immunoassay apparatus of the present invention has a function of monitoring a rotation timing at each one rotation of the non-magnetic plate 103 described above, and processing to add the magnetism signals acquired by rotating the non-magnetic plate 103 by plural times in the data collector described above by software, which is applied when the immunoassay with a high accuracy is carried out by using the function.

There is a case where distances between the respective inspection samples and the MR sensor 104 are not uniform in measuring by a small strain of the non-magnetic plate described above in fabrication, or bending in rotating the non-magnetic plate 103. Hence, the magnetic immunoassay apparatus of the present invention has a function of monitoring changes in displacements of positions of the non-magnetic plate 103 at which the respective inspection samples are installed in rotating, and correcting the magnetism signals from the respective inspection samples acquired by the measurement by using the displacement information by a software in the date collector described above.

The magnetic immunoassay apparatus of the present invention is arranged with the MR sensor described above such that the magnetism signal in a direction the same as a tangential line direction of the non-magnetic plate 103 described above is measured. A dispersed type waveform having a minimum value and a maximum value is obtained by detecting the magnetism signal from the inspection sample measured by the MR sensor 104 by the lock-in amplifier 109 described above. A difference between the maximum value and the minimum value in the dispersed type waveform (peak-to-peak intensity) is made to be an intensity of the magnetism signal for evaluating the inspection sample. A concentration of the antigen which becomes an inspection object is quantitatively evaluated from the change amount of the peak-to-peak intensity.

### Advantageous Effects of Invention

According to the present invention, there is realized an immunoassay system which can stably measure an antigen-antibody reaction without magnetic shielding and by a simple apparatus configuration.

### BRIEF DESCRIPTION OF DRAWINGS

FIG. 1 is a diagram showing a magnetic immunoassay apparatus using an AC magnetization measuring method of the present invention.

FIG. 2 is a view showing a magnetic marker configured by a magnetic particle, a polymer, and an antibody.

FIG. 3 is a view showing a coupling marker and an uncoupling marker in an inspection sample vessel in a case where an antibody is added to a bottom portion of the inspection sample vessel.

FIG. 4 is a diagram showing frequency dependencies of susceptibilities of a coupling marker and an uncoupling marker.

FIG. 5 is a view showing a coupling marker and an uncoupling marker in the inspection sample vessel in a case where an antibody is added to a polymer bead.

FIG. 6A is a diagram showing noise intensities from respective inspection vessels in a case where an excitation coil and an MR sensor of a magnetic immunoassay apparatus are separated.

FIG. 6B is a diagram showing noise intensities from respective inspection vessels in a case where an excitation coil and an MR sensor of a magnetic immunoassay apparatus are integrated.

FIG. 7 is a diagram showing a magnetic immunoassay apparatus using a difference by two MR sensors of the present invention.

FIG. 8A is a diagram showing a schematic diagram of a magnetism signal from an inspection sample vessel and measuring directions of respective MR sensors.

FIG. 8B is a diagram showing output waveforms of MR sensors arranged on an upper side and a lower side of an inspection sample vessel and a waveform by a difference between the respective MR sensors.

FIG. 9A is a diagram showing a signal from a magnetic marker detected by an MR sensor arranged on an upper side for an inspection sample.

FIG. 9B is a diagram showing a signal from a magnetic marker detected by an MR sensor arranged on a lower side for an inspection sample.

FIG. 9C is a diagram showing a waveform of subtracting the signal from the magnetic marker detected by the MR sensor arranged on the upper side from the signal of the magnetic marker detected by the MR sensor arranged on the lower side.

FIG. 10A illustrates diagrams showing an output of an MR sensor arranged on an upper side of a sample vessel (a), an output of an MR sensor arranged on a lower side of the sample vessel (c), and an observation result (b) of an output of a difference between the outputs of the respective sensors when a motor is not rotated.

FIG. 10B illustrates diagrams showing an output of an MR sensor arranged on an upper side of a sample vessel (a), an output of an MR sensor arranged on a lower side of the sample vessel (c), and an observation result (b) of an output of a difference between the outputs of the respective sensors when the motor is rotated.

FIG. 11 is a diagram showing a magnetic immunoassay apparatus having a displacement sensor for measuring a distance between an inspection sample and an MR sensor.

FIG. 12 is a diagram showing a change in a magnetism signal from a magnetism sensor for a distance between an MR sensor and a sample.

FIG. 13A is a diagram showing magnetism signal intensities from respective magnetic markers when the intensity is not corrected by distance information obtained by a displacement sensor (▴ plot) and when the intensity is corrected by the distance information ( plot).

FIG. 13B is a diagram showing a distance between an MR sensor and a sample in an inspection sample vessel.

FIG. 14 is a diagram showing a magnetism signal from a magnetic marker under a condition of setting a rotational speed as 8 rpm and a lock-in amplifier detection bandwidth as 53 Hz.

FIG. 15 is a diagram showing a magnetism signal from a magnetic marker under a condition of setting a rotational speed as 8 rpm and a lock-in amplifier detection bandwidth as 17 Hz.

FIG. 16 is a diagram showing a magnetism signal from a magnetic marker under a condition of setting a rotational speed as 8 rpm and a lock-in amplifier detection bandwidth as 5.3 Hz.

FIG. 17 is a diagram showing a magnetism signal from a magnetic marker under a condition of setting a rotational speed as 13 rpm and a lock-in amplifier detection bandwidth as 53 Hz.

FIG. 18 is a diagram showing a magnetism signal from a magnetic marker under a condition of setting a rotational speed as 13 rpm and a lock-in amplifier detection bandwidth as 17 Hz.

FIG. 19 is a diagram showing a magnetism signal from a magnetic marker under a condition of setting a rotational speed as 13 rpm and a lock-in amplifier detection bandwidth as 5.3 Hz.

FIG. 20 is a diagram showing a magnetism signal from a magnetic marker under a condition of setting a rotational speed as 26 rpm and a lock-in amplifier detection bandwidth as 53 Hz.

FIG. 21 is a diagram showing a magnetism signal from a magnetic marker under a condition of setting a rotational speed as 26 rpm and a lock-in amplifier detection bandwidth as 17 Hz.

FIG. 22 is a diagram showing a magnetism signal from a magnetic marker under a condition of setting a rotational speed as 26 rpm and a lock-in amplifier detection bandwidth as 5.3 Hz.

FIG. 23 is a diagram showing a rotational speed of a motor and a change in an SN ratio in a magnetism signal intensity from a magnetic marker in a detection bandwidth of a lock-in amplifier.

FIG. 24 is a diagram showing a measurement arrangement in a case where an excitation coil is placed horizontally.

### DESCRIPTION OF EMBODIMENTS

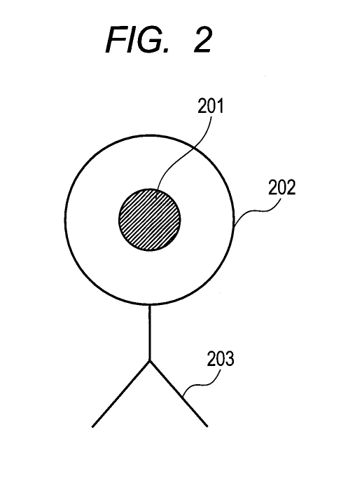

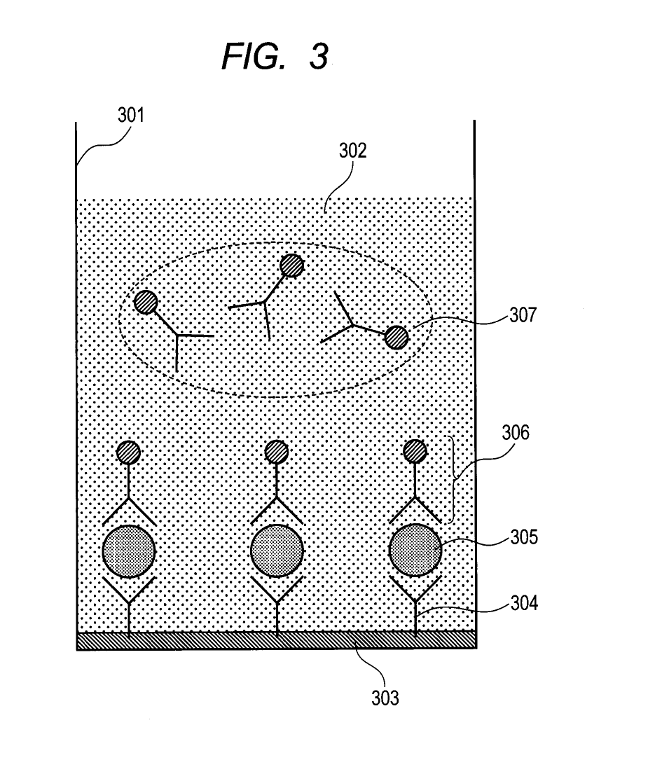

An explanation will be given of embodiments of the present invention in reference to the drawings as follows. According to the present invention, there is carried out a magnetic immunoassay using an AC magnetism as shown in FIG. 3 by using a magnetic marker consisting of a magnetic particle 201, a polymer 202, and an antigen for detection 203 as shown in FIG. 2.

An antigen 304 is fixed to a bottom portion 303 of an inspection sample vessel 301, and a magnetic marker is administered to the inspection sample vessel 301 into which an antigen 305 is put. At this occasion, there are respectively present a coupling marker 306 which is coupled with the antigen 304 by an antigen-antibody reaction in the inspection sample vessel and an uncoupling marker 307 depending on a concentration of the antigen 305 in an inspection solution 302. A size of the magnetic marker is in an order of 100 nm, and therefore, the magnetic marker is moved randomly and moved to rotate in the solution of the inspection sample vessel 301 by a thermal noise. The magnetic marker is configured by a magnetic particle, and therefore, the magnetic marker has a magnetic moment. An aggregate of the magnetic marker in the inspection sample vessel is totally magnetized by the magnetic moment, and the magnetization is attenuated exponentially over time. The relaxation phenomenon is referred to as Brawnian relaxation, and is proportional to a volume of the magnetic marker. A literature (B. Payet et al.: J. Magn. Magn. Mater. Vol. 186 (1998), p. 168.) shows that relaxation time τ by the Brawnian relaxation is expressed by τ=3 ηV/k_{B}T. Here, notation η designates a viscosity of the inspection solution, notation V designates the volume of the magnetic marker, notation k_{B} designates the Boltzman constant, and notation T designates a temperature of the inspection solution. Also, the volume V is expressed by V=(π/6)d³ by a diameter d of the magnetic marker. The above-described literature shows that an AC susceptibility of the magnetic marker consists of a real portion component χ′(ω)=[χ¹/{1+(ωτ) 2}]+χ∞, and an imaginary portion component χ″(ω)={(ωτχ¹)/1+(ωτ)²}. Here, a susceptibility component having a phase the same as a phase of an AC magnetism is a real portion component of an AC susceptibility which is generated at a magnetic marker when the magnetic marker is magnetized by the AC magnetism.

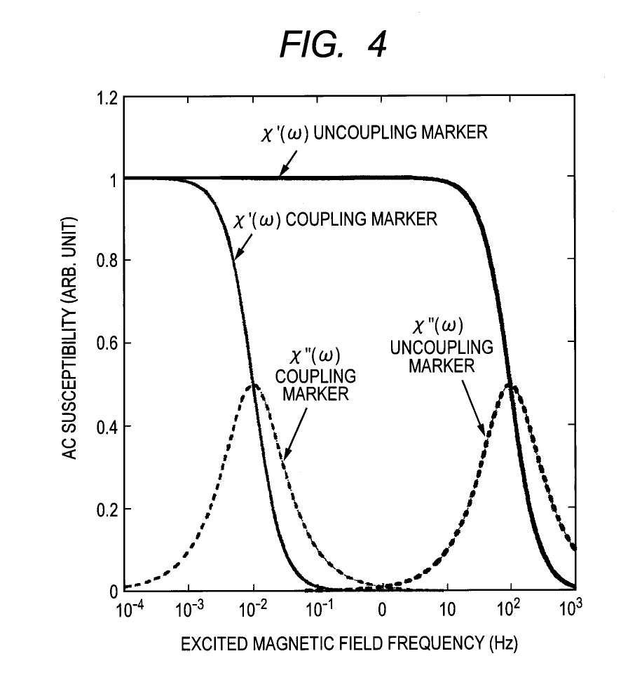

On the other hand, a susceptibility component having a phase shifted from a phase of an AC magnetism by 90° becomes an imaginary portion component. Based on the above-described, the coupling marker 306 and the uncoupling marker 307 significantly differ from each other in a size of the diameter d, and therefore, a significant difference is brought about between the relaxation time periods of the respective markers. A difference is shown in frequency dependency of the susceptibility as shown in FIG. 4 in the real portion component χ′ (ω) and the imaginary portion component χ″ (ω) of the AC susceptibility described above by the difference in the relaxation time. That is, the coupling marker shows a large susceptibility by an AC magnetism at a low frequency, and a sufficient susceptibility cannot be obtained at a high frequency. On the other hand, the uncoupling marker shows a sufficient susceptibility even at a high frequency. A frequency f of an AC magnetism at which the imaginary portion component χ″ (ω) of the susceptibility is expressed by f=1/(2πτ) as shown in FIG. 4. Therefore, a difference of the frequency dependency of the AC susceptibility between the coupling marker and the uncoupling marker can be applied to an immunoassay by using the difference.

An immunoassay is carried out from information of only the uncoupling marker which is efficiently obtained by applying an AC magnetism at several tens Hz or higher to an inspection sample. In the immunoassay, an AC susceptibility signal obtained from the inspection sample into which the antigen 305 is not put is defined as a reference signal B₀. With regard to an AC susceptibility signal B′ from an inspection sample in a case where the antigen is put into the inspection sample, the AC susceptibility signal B′ from the inspection sample is reduced from the reference signal B₀ described above by reducing the uncoupling marker 307 in comparison with the uncoupling marker 307 before administration of the antigen. A concentration of the antigen is quantitatively evaluated by a magnitude of the change amount α(α={B0−B′)/B0}×100 [%]).

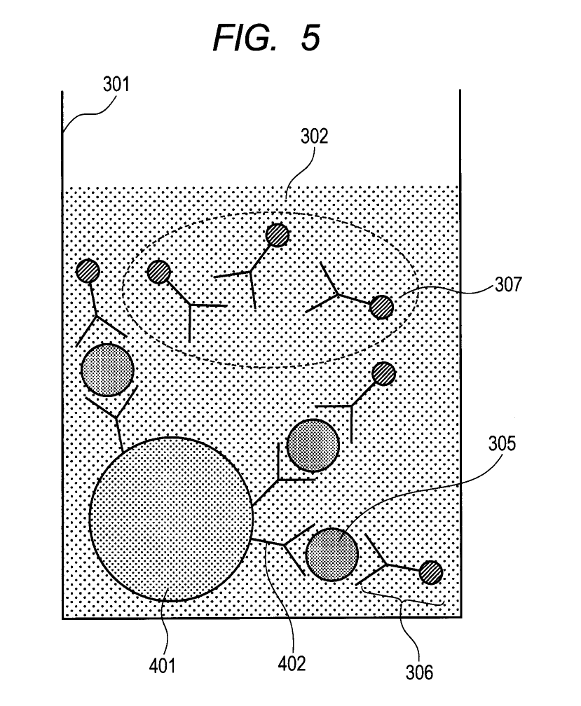

Although the coupling marker 306 described above is obtained by fixing the antigen at the bottom portion of the inspection sample vessel, there can also be used a polymer bead added with an antigen in place of the fixed antigen at the bottom portion (FIG. 5). In this case, there are respectively present the coupling marker 306 which is coupled with the polymer bead 401 by the antigen-antibody reaction and the uncoupling marker 307 in the inspection solution 302.

Incidentally, in a case of FIG. 5, a number of the magnetic markers adhered to the polymer beads 401 can be made to be larger than a number of the magnetic marker adhered to the bottom portion shown in FIG. 3.

### First Embodiment

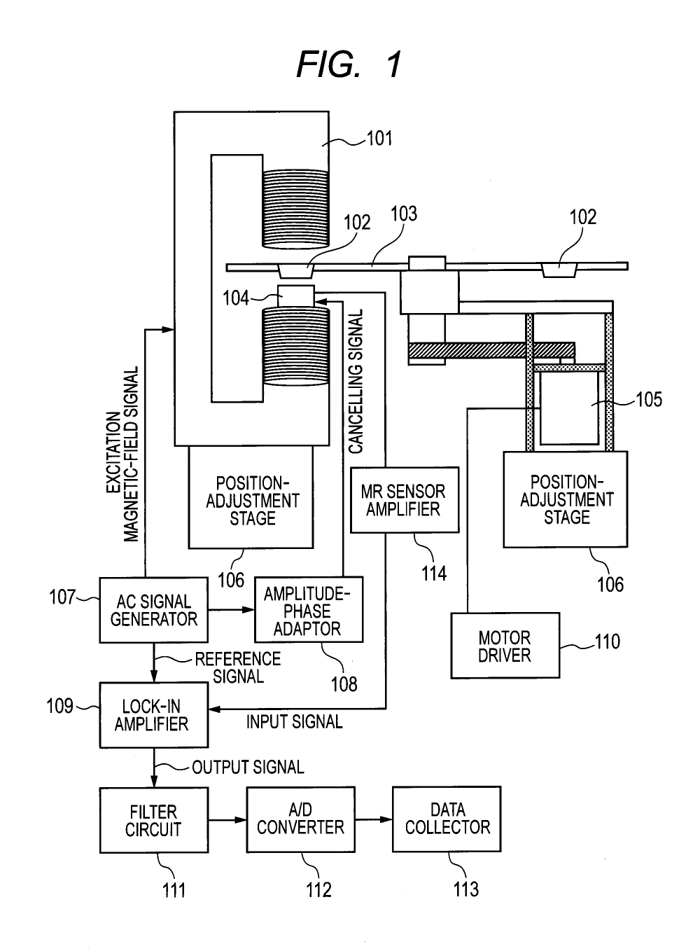

An explanation will be given of first embodiment of the present invention in reference to FIG. 1. An inspection sample is contained in an inspection vessel 102 included in the non-magnetic plate 103 as shown in FIG. 1. The non-magnetic plate 103 is rotated to move by a drive system configured by the DC motor 105. The inspection sample is magnetized by the AC magnetism from the excitation coil 101 by passing the non-magnetic plate 103 through the excitation coil 101 in rotating the non-magnetic plate 103. As shown in FIG. 1, the excitation coil 101 is of a Helmholtz coil type, and the inspection sample passes to traverse at a vicinity of a center between coils. The MR sensor 104 which measures a magnetism signal from the inspection sample is constructed by a structure integrated with the excitation coil 101. A system noise caused by a vibration can be reduced by integrating the excitation coil 101 and the MR sensor 104 in this way. According to the present embodiment, the non-magnetic plate 103 is configured by a shape of a circular disk, and 12 pieces of the inspection vessels 102 are aligned on the circular disk at a constant distance from a center of the circular disk, and aligned to be contiguous to each other while maintaining constant intervals thereamong. The inspection vessels aligned on the circular disk are successively attached with numbers from 1 to 12. In drawings used in the following explanation, attached vessel numbers correspond thereto. Incidentally, the numbers are successively attached irrespective of a right-handed order or a left-handed order.

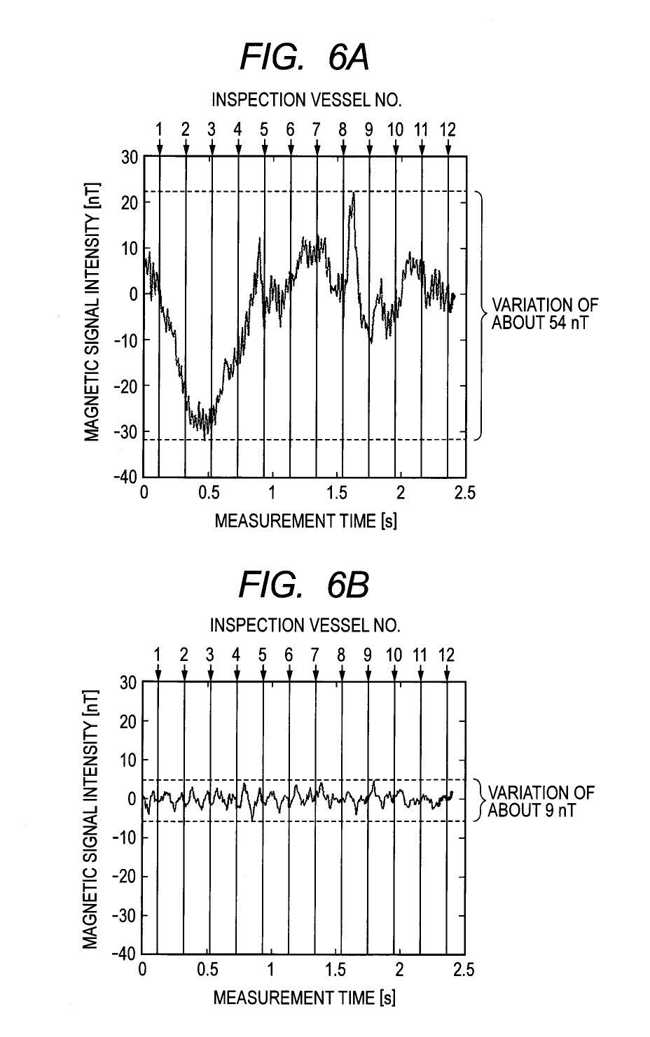

FIG. 6A and FIG. 6B show noise intensities from 12 pieces of the inspection vessels 102 into which the inspection samples are not put. Graduations on upper stages of FIGS. 6A and 6B indicate the inspection vessel numbers, and lower stages thereof indicate measurement time (a time period during which the measurement is carried out in passing the MR sensor 104 by rotating the circular disk described above). That is, there are present the respective inspection vessels 102 in correspondence with the inspection vessel numbers at portions of the FIGS. 6A and 6B indicated by longitudinal dotted lines.

FIG. 6A shows a case of a configuration of separating the excitation coil 101 and the MR sensor 104, and FIG. 6B shows a case of a configuration of the present invention integrating the excitation coil 101 and the MR sensor 104, respectively. A variation in the noise intensity can be reduced by about ⅙, and the noise intensities of the respective inspection vessels can be stabilized to the same degree by the integrated type structure according to the present invention. When the immunoassay is carried out, the AC magnetism for magnetizing the inspection vessels enters the MR sensor, and therefore, it is necessary to cancel a leak component of the AC magnetism entering the MR sensor.

According to the present invention, the leak component is canceled by outputting a magnetism having a phase inverse to a phase of the leak component of the AC magnetism to a small-sized coil incorporated in the MR sensor. When a position of the excitation coil or the MR sensor is varied by a vibration of a drive system or at a surrounding of the immunoassay apparatus in the canceling even by a small amount, the leak component that is canceled by the small-sized coil enters the MR sensor. Therefore, a remarkable variation is produced in the noise intensities from the respective inspection sample vessels as shown in FIG. 6A. On the other hand, even when the position of the excitation coil is varied, the MR sensor is varied also similarly by constructing the integrated type structure as shown in FIG. 6B. Therefore, the leak component which is canceled by the small-sized coil remains unchanged from that in canceling, and variations in the noise intensities from the respective inspection sample vessels can be restrained.

The leak component is canceled by optimally adjusting an amplitude and a phase of a signal inputted to the small-sized coil by an amplitude-phase adjustment. At that occasion, the leak component is canceled to a degree of not saturating an input unit of the lock-in amplifier 109 when an output of the MR sensor 104 is connected as an input signal of the lock-in amplifier. A change in a phase of a magnetism signal from the inspection sample is detected by the lock-in amplifier 109 by using an output of the AC signal generator which is a signal source of the AC magnetism for the reference signal of the lock-in amplifier 109. It is convenient to use a 2-phase lock-in amplifier which can simultaneously output a real portion component and an imaginary portion component of a detected signal without adjusting a phase for the lock-in amplifier 109 used. The frequency dependencies of the AC susceptibilities of the coupling marker and the uncoupling marker differ from each other as shown in FIG. 4.

Hence, since the immunoassay apparatus of the present invention uses the uncoupling marker, the frequency band of the AC magnetism in a range of 10 Hz through 1 kHz is used. It is further optimal to a use a frequency band of an AC magnetism of about 100 through 500 Hz in consideration of a surrounding environmental magnetism noise, a 1/f noise characteristic of the MR sensor, a white noise level, and an intensity of a magnetism signal from the uncoupling marker.

### Second Embodiment

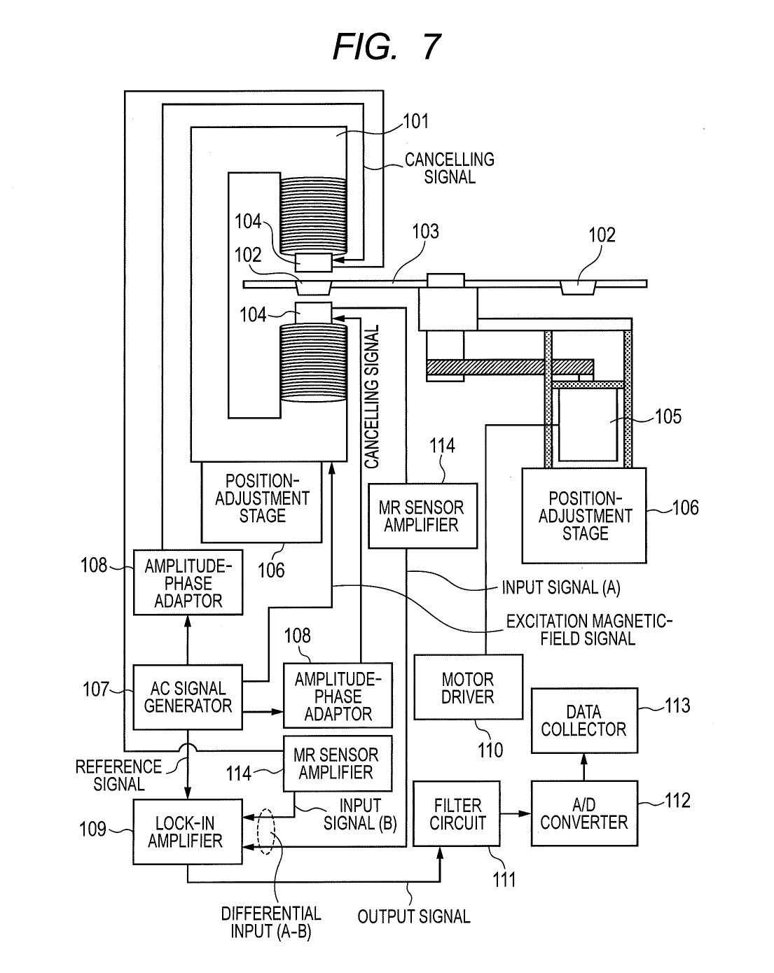

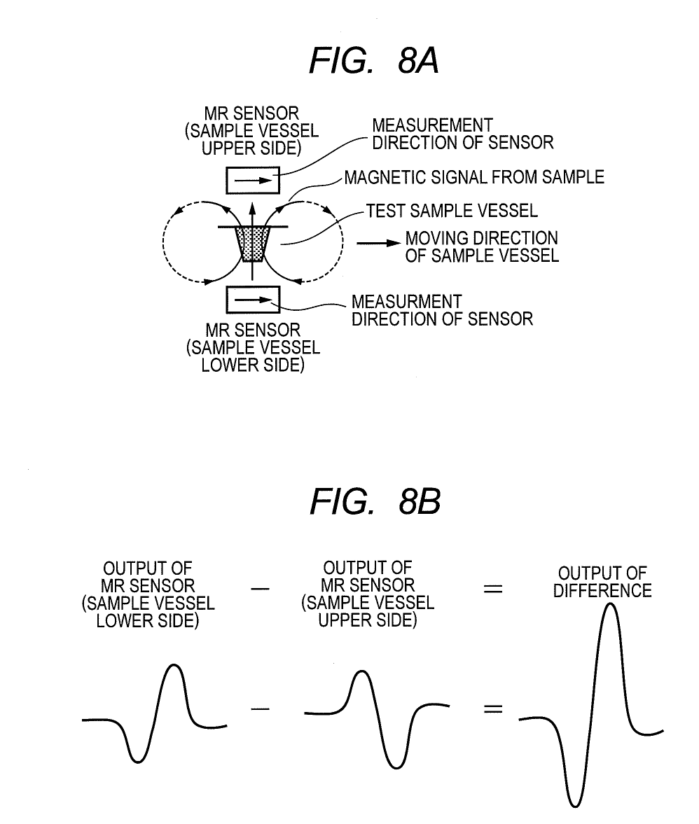

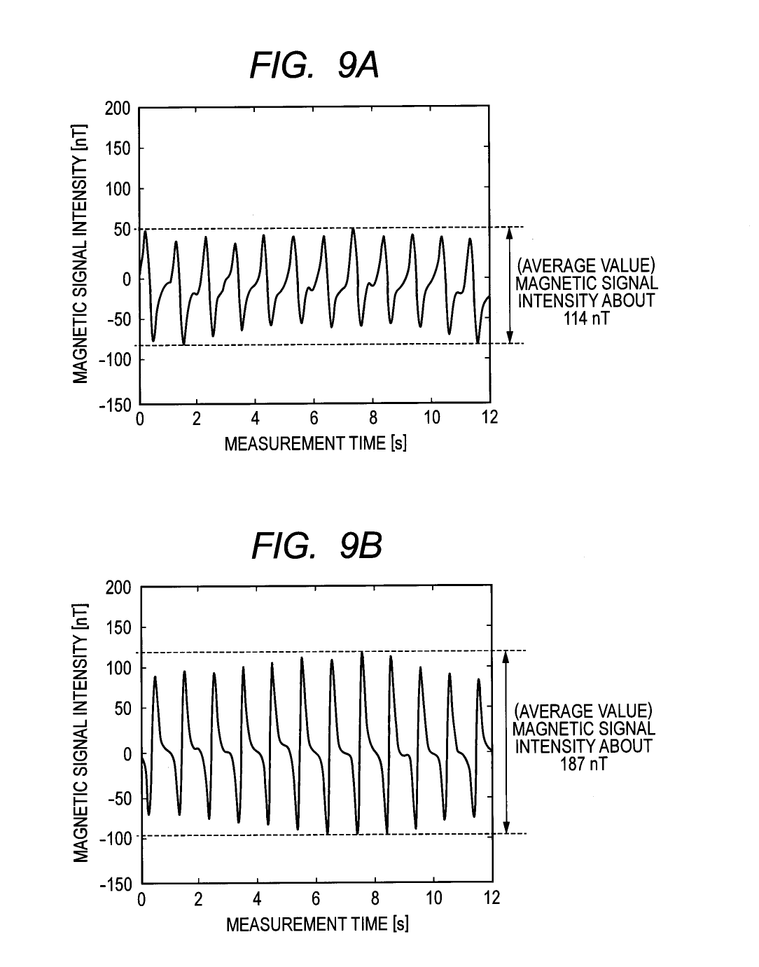

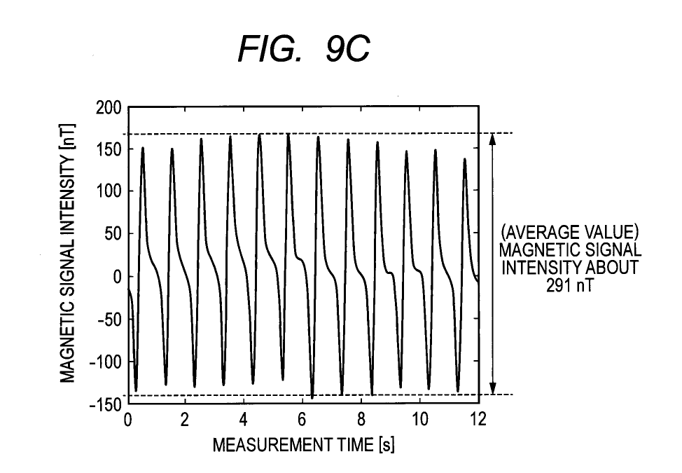

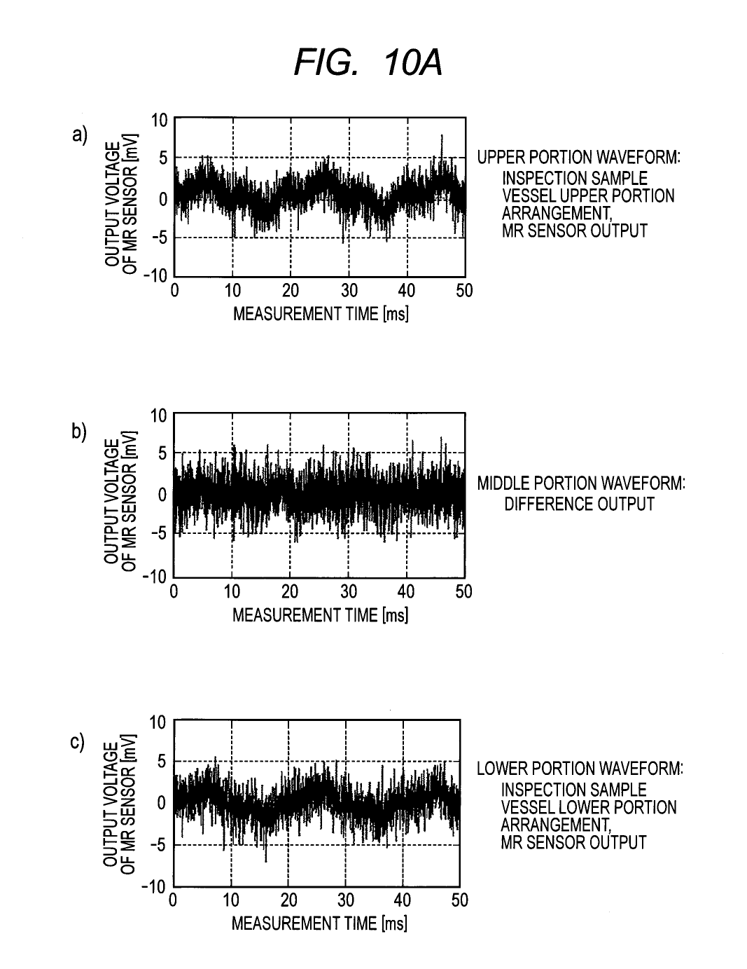

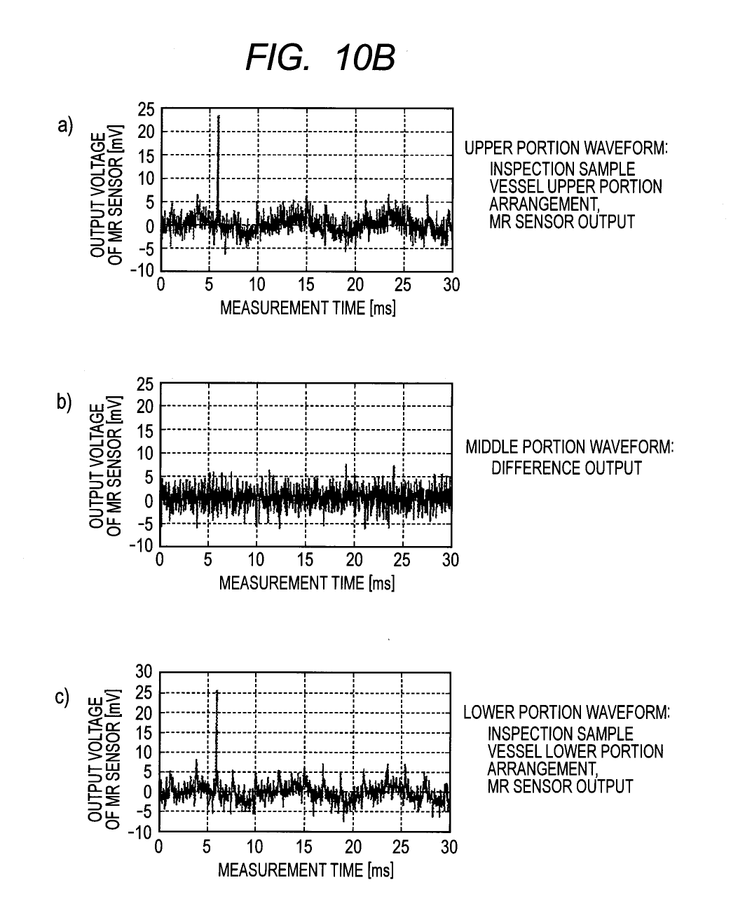

An explanation will be given of a second embodiment of the present invention in reference to FIG. 7. There is constructed a configuration of using two of the MR sensors 114 and interposing the inspection sample vessel by the respective MR sensors 114 as shown in FIG. 7. The inspection sample vessel 102 passes through the excitation coil 101 by rotating the non-magnetic plate 103 by using the drive unit configured by the DC motor 105 similar to the first embodiment. At that occasion, the inspection sample in the inspection sample vessel 102 is magnetized by the AC magnetism from the excitation coil 101 (FIG. 8A). Magnetism signals from the magnetized inspection sample are configured by dispersion type waveforms (waveforms having minimum values and maximum values) respectively inverted by the MR sensor 104 arranged at an upper portion of the inspection sample vessel 102 and the MR sensor 104 arranged at a lower portion thereof (FIG. 8B). Therefore, it can said that the magnetism signal intensity is increased more than before calculating a difference between output signals of the respective MR sensors 104 by detecting a change in a phase of a magnetism signal which is produced by calculating the difference between the output signals of the respective MR sensors 104 by the lock-in amplifier 109 (FIG. 8B). Incidentally, magnetism measuring directions of the respective MR sensors 104 are in the same direction, and in parallel with a tangential direction of the non-magnetic plate 103. FIGS. 9A and 9B, and 9C show results of magnetism signal waveforms and magnetism signal intensities in a case of using the MR sensor (input signal B) 104 which is arranged at an upper portion of the inspection sample vessel 102, in a case of using the MR sensor (input signal A) 104 which is arranged at a lower portion of the inspection sample vessel 102, and in a case of calculating a difference between outputs of the respective MR sensors 104 (input signal A−input signal B) for detecting magnetism signals from 12 pieces of the inspection sample vessels 102 provided at the non-magnetic plate 103 in which the same magnetic markers are put into the inspection sample vessels 102. It is known that the magnetism signal waveforms provided from the respective MR sensors 104 are inverted as described as FIG. 8B (FIGS. 9A and 9B). Also, the magnetic signal intensity is increased more than that before calculating the difference (average magnetism signal intensity; 187 nT) by about 1.7 times by calculating the difference (average magnetism signal intensity: 291 nT) by using the respective MR sensors 104. The difference calculating processing not only increases the magnetism signal intensity from the inspection sample but can reduce the environmental magnetism noise which enters the MR sensors 104 with the same phase (FIGS. 10A and 10B). FIGS. 10A and 10B show results of monitoring outputs of the respective MR sensors 104 and an output of calculating the difference which are measured in a state where the inspection sample is not present by an oscilloscope. It is known that in a case where the DC motor is not rotated, a line noise (50 Hz component and a harmonic component thereof) which is the environmental noise enters the respective MR sensors with the same phase, and the line noise can be canceled by calculating the difference (FIG. 10A). Although when the DC motor is rotated, a magnetism noise enters the respective MR sensors 104 remarkably from the DC motor, the magnetism noise can be reduced by calculating the difference (FIG. 10B).

That is, magnetism signals from the inspection sample can be measured with a high SN ratio and clearly by a configuration of calculating a difference of the magnetic signals from the inspection sample by using the two MR sensors in the immunoassay apparatus according to the present invention.

### Third Embodiment

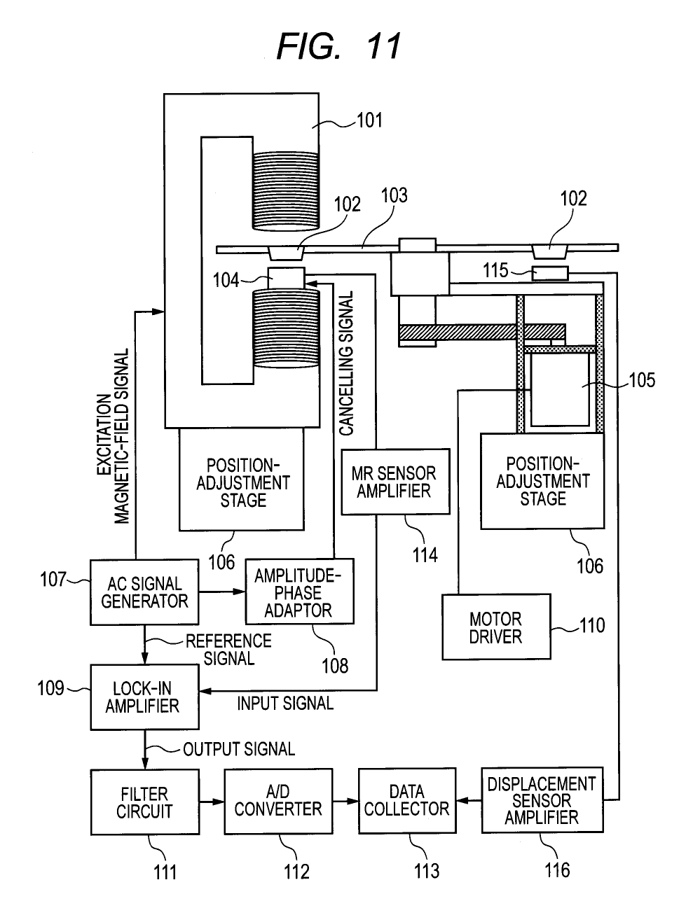

A third embodiment of the present invention includes an optical type displacement sensor for monitoring a displacement between the MR sensor and the inspection sample vessel in the immunoassay apparatus described in the first embodiment or the second embodiment of the present invention. As shown in FIG. 11, the optical type displacement sensor 115 is arranged right below the inspection sample vessel 102 provided at the non-magnetic plate 103. A magnetism of a magnetism signal from the inspection sample is measured by the MR sensor 104, at the same time, a change in the displacement of the inspection sample vessel by bending the non-magnetic plate in rotating the non-magnetic plate is simultaneously measured by the optical type displacement sensor. Incidentally, despite the simultaneous measurement, the MR sensor and the optical type displacement sensor detect respectively separate pieces of information of the magnetism signal and the displacement of the inspection sample in view of a relationship of arranging the MR sensor and the optical type displacement sensor. There is a change in the magnetic signal intensity by a change in the distance between the MR sensor and the inspection sample vessel as an influence of the change in the displacement of the inspection sample vessel which is effected on the magnetism measurement.

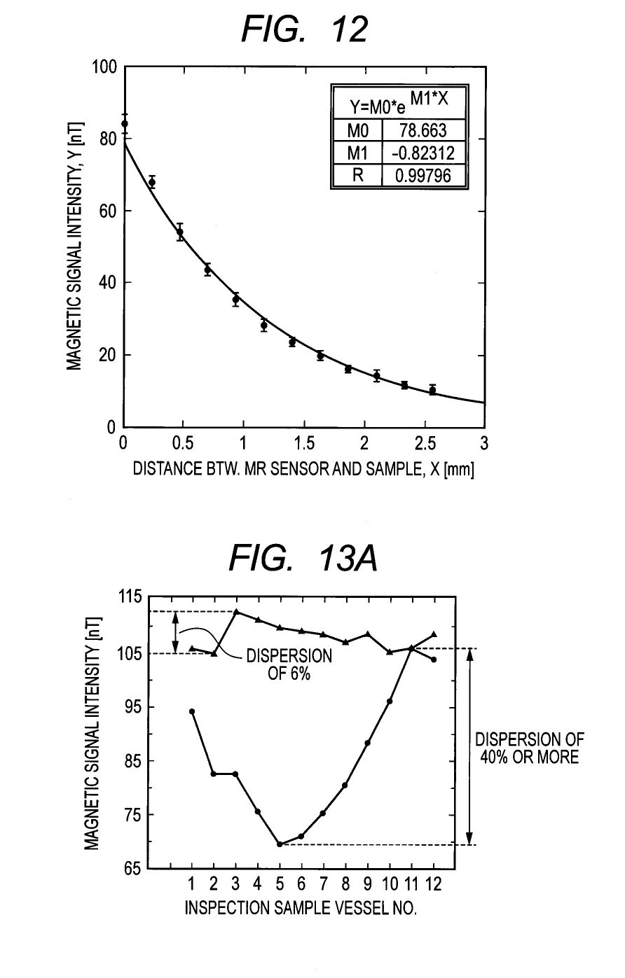

FIG. 12 shows a change in a magnetism signal intensity when magnetism markers are administered to 12 pieces of the inspection sample vessels provided at the non-magnetic plate, and a distance between the MR sensor and the inspection sample vessel is changed by every 0.23 mm within a range from 0 to 2.56 mm. Incidentally, plotting of FIG. 12 shows an average value of 12 samples. Here, distance 0 indicates a position of separating the MR sensor and the inspection sample vessel by 1 mm at which the MR sensor and the inspection sample vessel are proximate to each other the most. As measurement conditions, an excitation magnetic field intensity is 0.4 mT, and an excitation magnetic field frequency is 150 Hz. It is known from FIG. 12 that when the distance is increased, the magnetism signal intensity is reduced, and when the distance is increased by 2.63 mm, the magnetism signal intensity from the magnetic marker used is reduced by about 88%. A bold line in FIG. 12 shows an attenuation curve relative to a change of the magnetism signal intensity which is fitted by an exponential function by the distance. Incidentally, fitting parameters are shown in a table on the right upper side of the drawing. The abscissa of the drawing is indicated by X and the ordinate is indicated by Y. According to the present measurement, a time constant M1 of the attenuation curve is about 0.8. It is necessary that the distance between the MR sensor and the sample is constant in order to realize table magnetism measurement as described above.

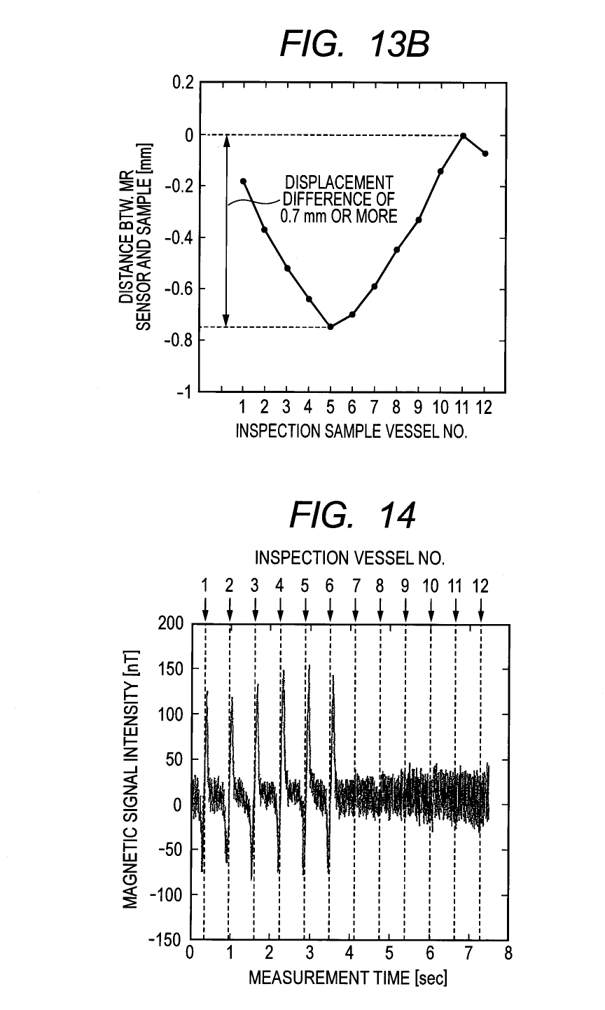

However, even when the non-magnetic plate is stably rotated actually with a fine mechanical accuracy, it is difficult to nullify a variation in a displacement equal to or less than 0.1 mm in the non-magnetic plate. There is also a factor of increasing a variation in a displacement by bending owing to an individual difference that is brought about in fabricating the non-magnetic plate. Therefore, the change in the magnetism signal intensity by the variation in the displacement can be resolved by correcting the magnetism signal by the displacement information by measuring the displacement simultaneously with the magnetism measurement as described above. FIG. 13 shows a result of measuring the magnetism signal intensities from the respective inspection samples and the changes in the displacements of the respective inspection sample vessels by administering the magnetic markers to 12 pieces of the inspection sample vessels provided at the non-magnetic plate. Despite the use of the same magnetic marker, there is a variation of the magnetism signal intensity from the inspection sample among the inspection sample vessels, and there is brought about a difference equal to or more than about 20% at maximum ( plotting in FIG. 13A). A distance between the MR sensor and the inspection sample differs among the inspection sample vessels, and there is a difference equal to or more than 0.7 mm at maximum (FIG. 13B). It is known that with regard to a dispersion in the magnetism signal intensity and the dispersion in the distance among the inspection sample vessels, not only the inspection sample vessel (vessel 11) at which changes in respective physical quantities (magnetism signal intensity and distance) are maximized and the inspection sample vessel (vessel 5) at which the changes are minimized stay the same, but patterns of changing the respective physical quantities among the inspection sample vessels are similar ( plotting in FIG. 13A and FIG. 13B).

▴ plotting of FIG. 13A shows a result of correcting the magnetism signal intensities from the respective inspection sample vessels by the change amount of the distance between the MR sensor and the sample by using the attenuation curve provided at FIG. 12. It is known in the connection that the variation in the magnetism signal intensities among the inspection sample vessels which has been equal to or more than 40% at maximum is improved to about 6% from FIG. 13A in which a reference is set by the vessel 11 at which the magnetism signal intensity is maximized.

The variation in the magnetism signal intensity by the system noise caused by the change in the distance is considerably reduced by correcting the distance between the MR sensor and the sample according to the present invention in the immunoassay apparatus as described above.

### Fourth Embodiment

A fourth embodiment of the present invention realizes stable magnetism measurement without magnetic shielding from conditions of the rotational speed of the non-magnetic plate and the bandwidth of the lock-in amplifier in the immunoassay apparatus described in any of the first through the third embodiments of the present invention. An AC magnetization measuring method according to the present invention uses the lock-in amplifier in order to obtain a weak magnetism signal from the inspection sample embedded in the noises. Therefore, it seems that the noise mixed to the magnetism signal can significantly be reduced when the bandwidth of detecting lock-in is pertinently set in the lock-in amplifier. However, although the noise is reduced by narrowing the bandwidth, the magnetism signal is reduced depending on the speed of the non-magnetic plate into which the inspection sample is put. Therefore, the SN ratio of the magnetism signal cannot be improved by simply narrowing the bandwidth of the lock-in amplifier in consideration of a total balance.

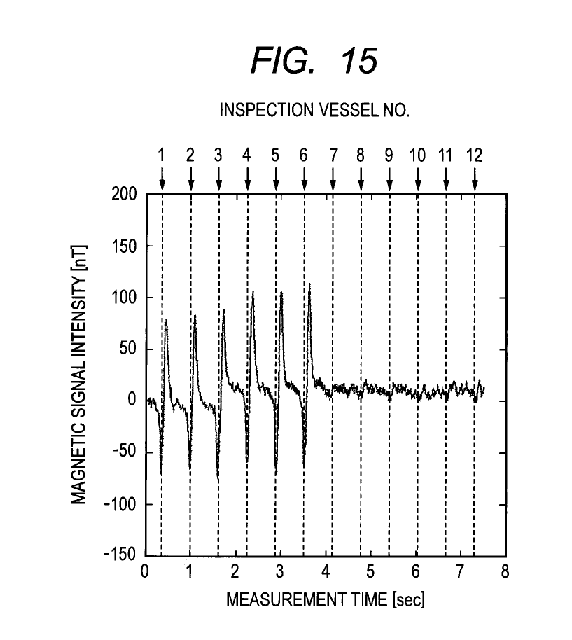

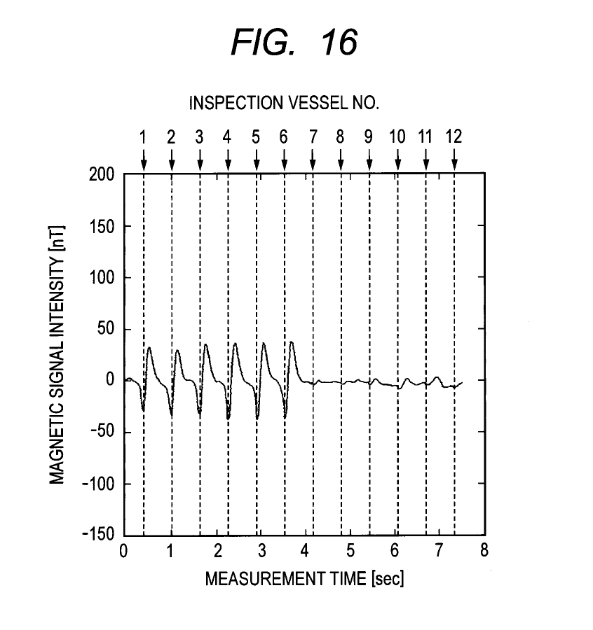

Hence, according to the present invention, there is made a specification of capable of rotating the non-magnetic plate at a low speed such that the magnetism signal intensity from the inspection sample is not lowered even in a state of narrowing the bandwidth of the lock-in amplifier. There is used a geared DC motor mounted with a small-sized gear at an inner portion of the motor such that the non-magnetic plate can be rotated at a low speed easily down to about 1 rpm. There is constructed a configuration of arranging the motor and the MR sensor remotely from each other by driving the motor by a belt without directly connecting the motor and the non-magnetic plate in order to reduce an influence of a magnetism noise from the motor entering the MR sensor. FIGS. 14 through 22 show a result of measuring magnetism signals from the respective inspection sample vessels by putting 6 samples of the magnetic markers into 12 pieces of the measurement sample vessels provided at the non-magnetic plate and emptying the remaining vessels. The bandwidth is set to 5.3 Hz, 17 Hz, and 53 Hz, and the rotational speed is set to 8 rpm, 13 rpm, and 26 rpm respectively in order to investigate influences of the bandwidth of the lock-in amplifier and the rotational speed of the non-magnetic plate effected on the magnetism signal.

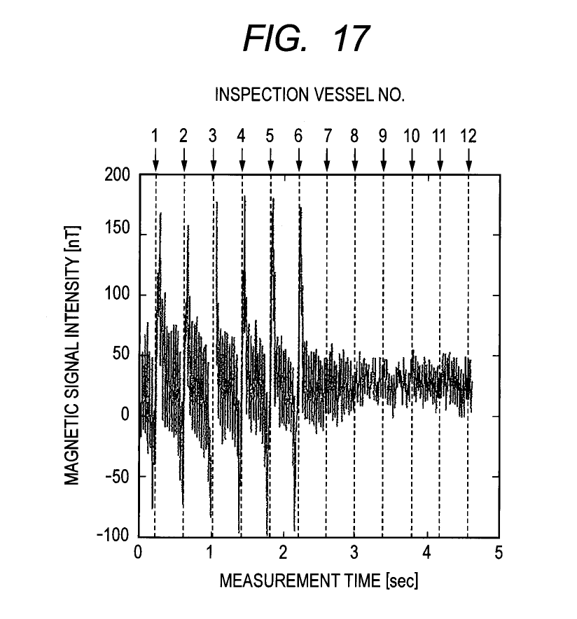

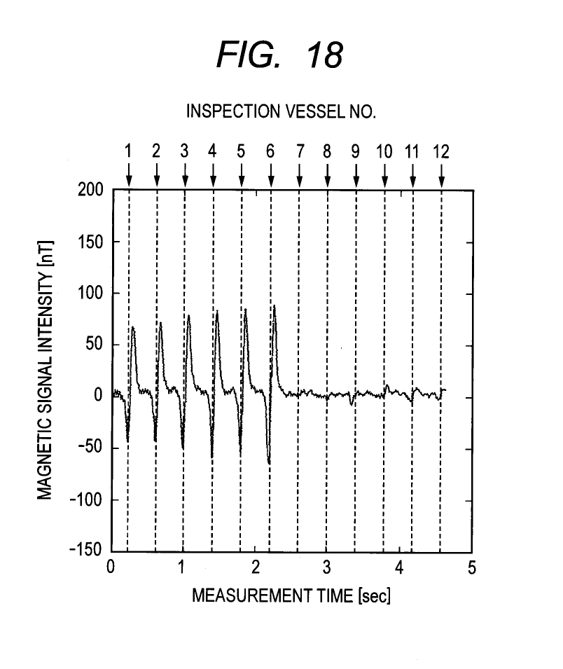

When drawings and measuring conditions are corresponded to each other, the rotational speed is 8 rpm in any of FIGS. 14 through 16, and the bandwidth is 53 Hz, 17 Hz, or 5.3 Hz successively from FIG. 14. Next, the rotational speed is 13 rpm in any of FIGS. 17 through 19, and the bandwidth is 53 Hz, 17 Hz, or 5.3 Hz successively from FIG. 17. Next, the rotational speed is 13 rpm in any of FIGS. 17 through 19, and the bandwidth is 53 Hz, 17 Hz, or 5.3 Hz successively from FIG. 17. The rotational speed is 26 rpm in any of FIGS. 20 through 22, and the band width is 53 Hz, 17 Hz, or 5.3 Hz successively from FIG. 20.

Excitation magnetic field conditions (excitation magnetic field frequency and excitation magnetic field intensity) are made to be 120 Hz and 1 mT, and the measurement is carried out by the configuration of the immunoassay apparatus shown in FIG. 1. A rotational speed of the non-magnetic plate is 8 rpm, 13 rpm, or 26 rpm, and therefore, a time period of rotating the plate by one rotation is 7.5 sec, 4.6 sec, and 2.3 sec, respectively. FIGS. 14 through 22 show a result of processing to add magnetic signals of an amount of 25 rotations, and the respective inspection sample vessels are disposed at dotted line portions in the longitudinal direction in the graph. In view of FIGS. 14 through 22, when the bandwidth is increased in all of the rotational speeds, a variation in the noise intensity (empty vessels 7 through 12) is reduced. In a case where the rotational speed is 8 rpm, there is provided a dispersion type shape (shape having a minimum value and a maximum value) in which the magnetism signal waveform from the sample is clear in all of band widths (FIGS. 14 through 16).

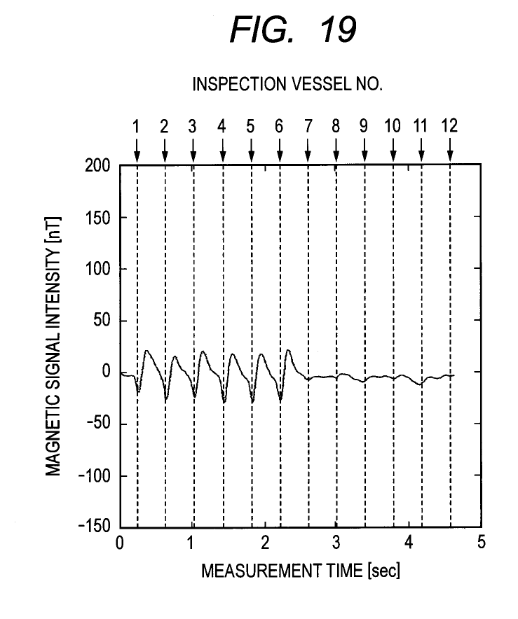

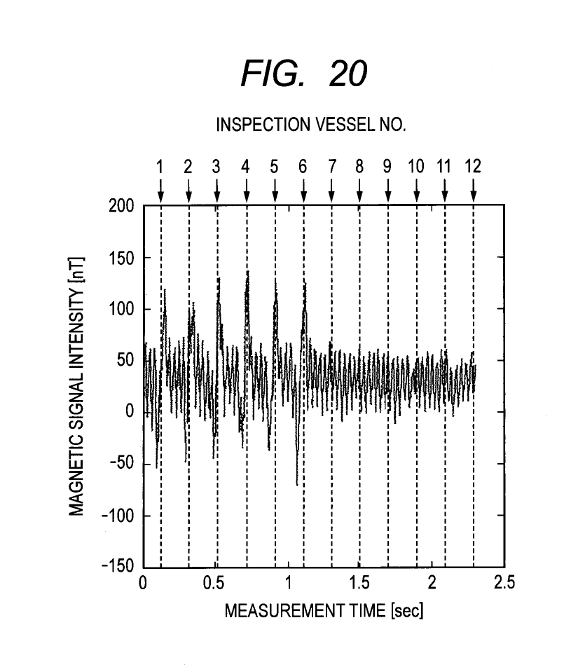

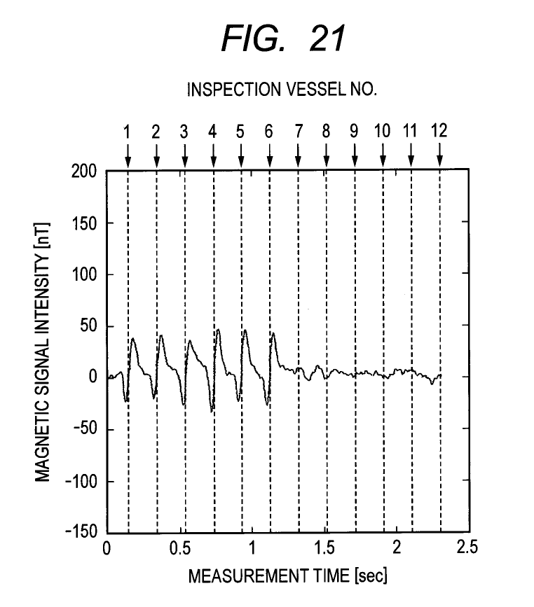

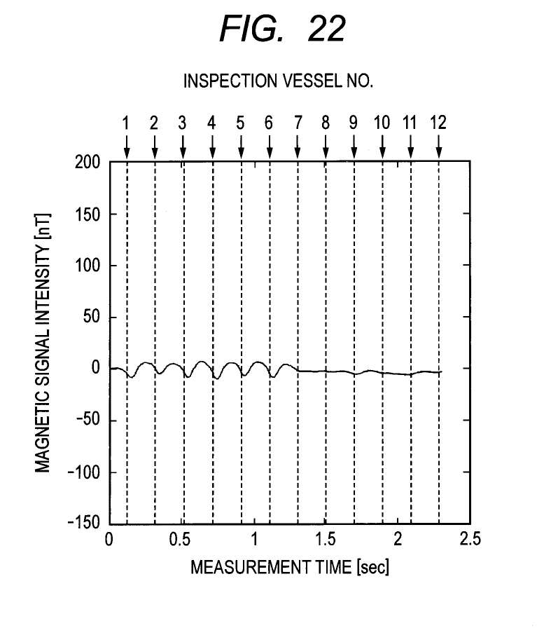

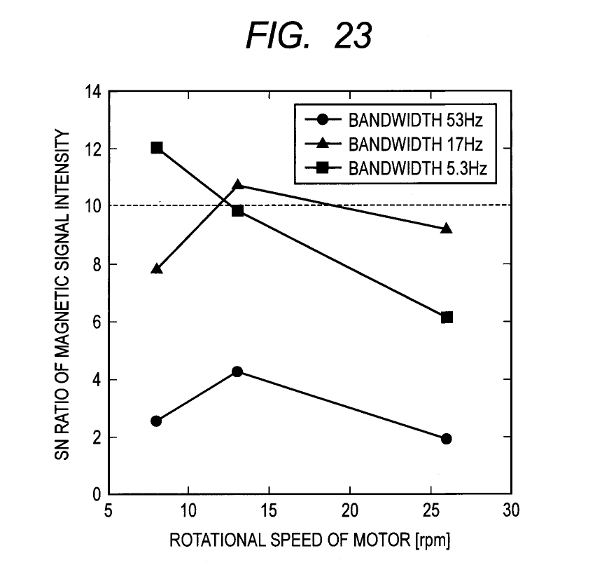

On the other hand, there is observed a change in the shape of the magnetism signal waveform from the inspection signal with the bandwidth of 53 Hz in the case of the rotational speed of 13 rpm (FIG. 19). There is brought about a change in the shape of the magnetism signal from the sample with the bandwidth of 17 Hz in the case of the rotational speed of 26 rpm (FIG. 21), and a significant change in a shape is shown at the bandwidth of 5.3 Hz (FIG. 22). FIG. 23 shows SN ratios of magnetism signals under conditions of respective band widths and the respective rotational speeds by using the measurement data of FIGS. 14 through 22. Here, there are respectively used the magnetic signal intensity from the inspection signal into which the magnetic marker is put and a noise intensity from an empty vessel in order to obtain the SN ratios.

FIG. 23 shows an SN ratio (S/N=10) when the immunoassay apparatus is covered by magnetic shielding, and a supersonic motor is used in the drive system by a dotted line. As shown in FIG. 23, the SN ratio is the lowest and is about 4 or less under a condition of the band width of 53 Hz in all of the rotational speeds ( plotting in FIG. 23). The dependency of the SN ratio on the rotational speed shows similar changes at the bandwidths of 53 Hz and 17 Hz ( plotting, ▴ plotting in FIG. 23).

On the other hand, an increase in the SN ratio is caused along with a reduction in the rotational speed under a condition of the bandwidth of 5.3 Hz, and the SN ratio is maximized to about 12 at the rotational speed of 8 rpm) (▪ plotting in FIG. 23). As described above, according to the present invention, there is provided the immunoassay apparatus which realizes a function of a level the same as a level of a condition of using the magnetic shielding and the ultrasonic motor by optimally setting the bandwidth of the lock-in amplifier and the rotational speed of the non-magnetic plate.

### Fifth Embodiment

A magnetism measuring direction of the MR sensor is set as follows in order to be able to read the magnetism signal obtained by the magnetism measurement easily in the immunoassay apparatus described in any of the first embodiment through the fourth embodiment of the present invention. The MR sensor is installed such that the magnetism measuring direction of the MR sensor is in parallel with a tangential direction of a circumference when the non-magnetic field is moved to rotate. For example, the magnetism signal from the sample in the tangential direction of the circular disk is measured by the MR sensor at a position of viewing the non-magnetic plate from right above the disk in a case where the non-magnetic plate is a circular disk. In this way, the magnetic signal from the sample is detected by a dispersion type shape (shape showing a minimum and a maximum) as shown in FIG. 9 or FIGS. 14 through 22 by setting the magnetism measuring direction of the MR sensor. Therefore, when such a signal shape can be obtained, since values of the minimum and the maximum are clear, an accurate evaluation can be carried out in the inspection by setting the magnetism signal intensity from the sample by a sum of the minimum value and the maximum value. On the other hand, in a case where the magnetism signal from the sample in a direction orthogonal to the tangential direction is measured by the MR sensor, the magnetism signal from the sample is configured by a single peak shape having only the minimum value or the maximum value. In that case, an evaluation is carried out by the peak value in the inspection.

### Sixth Embodiment

The non-magnetic plate may be moved linearly in the immunoassay apparatus described in any of the first embodiment through the fourth embodiment of the present invention. In that case, the magnetism measuring direction of the MR sensor is set in parallel with the moving direction of the non-magnetic plate. In this way, the magnetism signal from the sample is detected by a dispersion type shape (shape showing a minimum and a maximum) as shown in FIG. 9 or FIGS. 14 through 22 by setting the magnetism measuring direction of the MR sensor similar to fourth embodiment. Although one time of the movement will do in a case of magnetism measurement in real time, in a case where a highly accurate inspection is carried out, the adding processing is carried out by iteratively carrying out the linear movement. At that occasion, in a case where there is provided a magnetism signal of a dispersion shape showing first a minimum and next a maximum in a first movement (first time movement), there is provided the magnetism signal of a dispersion shape showing first a maximum and next a minimum in a case of successive returning linear movement (second time movement) by iterative linear movement. Therefore, in a case of carrying out the adding processing, for example, the addition is carried out by inverting either of magnetism signals obtained by an odd number time or an even number time movement.

### Seventh Embodiment

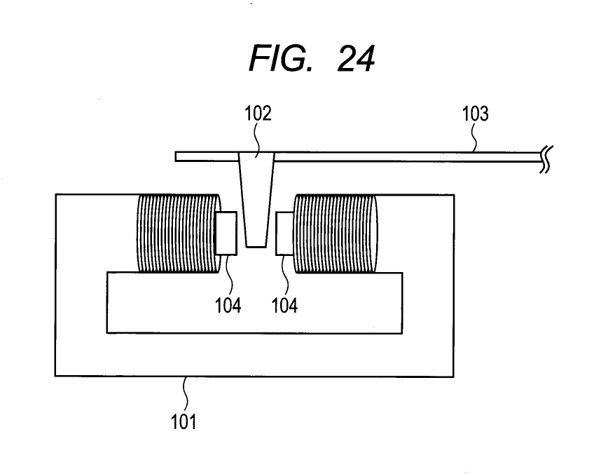

In a case where there is a depth in the inspection sample vessel installed at the non-magnetic plate in the immunoassay apparatus described in any of the first embodiment through the sixth embodiment of the present invention, the case can be dealt with by horizontally placing the excitation coil (FIG. 24). As shown in FIG. 24, the magnetism is measured by the MR sensor from a side face of the inspection sample vessel by using the MR sensor installed at the excitation coil arranging the inspection sample vessel horizontally. FIG. 24 shows an example of capable of similarly applying the difference calculating processing of the respective MR sensors as described above in the second embodiment by using two of the MR sensors.

### Eighth Embodiment

There is used the Helmholtz type coil type described in FIGS. 1, 7, 11, and 24 for the excitation coil used for applying a uniform AC magnetic field to the sample in the immunoassay apparatus described in any of the first through the seventh embodiments. At that occasion, the excitation coil becomes a closed circuit of a magnetic flux when the excitation coil is configured by a shape having a structure which is a continuous at other than a gap between the coils through which the sample passes, and a metal having a high permeability is used for a core member of the excitation coil. Thereby, the leak magnetic flux of the magnetic field from the excitation coil can be reduced. A channel shape or the like can be applied as an example of the shape of the excitation coil. The core member of the coil can be used even in a specification in which a magnetic material is not used. The excitation coil may be configured not by the Helmholtz coil type but by a simple coil configured only by one side thereof.

### Ninth Embodiment

It is further effective when only a main body portion of the motor is covered by a magnetic material having a high permeability of permalloy or the like for reducing a magnetic noise emitted from the DC motor in the immunoassay apparatus described in any of the first embodiment through the eighth embodiment. In a case where the DC motor used includes a brush at an inner portion thereof, the motor is rotated by mechanical contact between the brush and a commutator. Therefore, there is a case of emitting an electric noise by making a spark current flow in the mechanical contact. In that case, the electric noise caused by the spark current is reduced by connecting an electronic part of a capacitor, a varistor, a choke coil or the like at a terminal portion of the motor. In a case of using a brushless DC motor which does not have a brush at an inner portion of the motor, an influence of the electric noise caused by the spark current can be avoided.

### LIST OF REFERENCE SIGNS

101 . . . excitation coil, 102 . . . inspection sample vessel, 103 . . . non-magnetic plate, 104 . . . MR sensor, 105 . . . DC motor, 106 . . . position-adjustment stage, 107 . . . AC signal generator, 108 . . . amplitude-phase adjustor, 109 . . . lock-in amplifier, 110 . . . motor driver, 111 . . . filter circuit, 112 . . . A/D converter, 113 . . . data collector, 114 . . . MR sensor amplifier, 115 . . . displacement sensor, 116 . . . displacement sensor amplifier, 201 . . . magnetic particle, 202 . . . polymer, 203 . . . antibody, 301 . . . vessel for inspection, 302 . . . inspection sample solution, 303 . . . fixed board added to bottom portion of vessel for inspection, 304 . . . antibody added to fixed board, 305 . . . antigen, 306 . . . coupling marker, 307 . . . uncoupling marker, 401 . . . polymer bead, 402 . . . antigen adhered to polymer bead

1. A magnetic field measuring apparatus of measuring a state of an antigen in an inspection sample by an antigen-antibody reaction by using a magnetic marker as a mark configured by a magnetic particle, the magnetic field measuring apparatus comprising:
an inspection sample vessel for containing the magnetic marker and the inspection sample;
an excitation coil of applying an AC magnetic field to the inspection sample contained in the inspection sample vessel; and
a magnetism sensor for measuring a magnetism signal emitted from the inspection sample applied with the AC magnetic field;
wherein the magnetism sensor is configured by a structure integrated with the excitation coil for reducing a system noise caused by a vibration of the apparatus.

2. The magnetic field measuring apparatus according to claim 1, wherein the excitation coil is configured by a pair of excitation coils opposed to each other, the magnetism sensor is arranged to be opposed to one of the pair of excitation coils, and the magnetism sensor is arranged to be integrated with at least one of the excitation coils.

3. The magnetic field measuring apparatus according to claim 1, further comprising:
a non-magnetic plate mounted with the inspection sample vessel; and
a drive system configured by including a motor for linearly moving or moving to rotate the non-magnetic plate;
wherein an AC magnetic field is applied to the inspection sample by using the excitation coil while rotating the non-magnetic plate.

4. The magnetic field measuring apparatus according to claim 3, further comprising:
a lock-in amplifier for detecting a change in a phase of the magnetism signal from the inspection sample detected by the magnetism sensor.

5. The magnetic field measuring apparatus according to claim 3, wherein in a case of linearly moving the non-magnetic plate, a magnetism measuring direction of the magnetism sensor is in parallel with a moving direction of the non-magnetic plate; and
wherein in a case of moving to rotate the non-magnetic plate, a tangential direction in a circumference of rotation and a magnetism measuring direction of the magnetism sensor are in parallel with each other.

6. The magnetic measuring apparatus according to claim 3, wherein the excitation coil is arranged such that a line of a magnetic force generated at a vicinity of a center of the excitation coil is in a direction intersecting with a main surface of the non-magnetic plate.

7. The magnetic measuring apparatus according to claim 3, wherein the excitation coil is arranged such that a line of a magnetic force generated from a vicinity of a center of the excitation coil is substantially in parallel with a main surface of the non-magnetic plate.

8. The magnetic measuring apparatus according to claim 4, wherein two of the magnetism sensors are included;
wherein the respective magnetism sensors are arranged to be integrated with the pair of respective excitation coils;
wherein the inspection sample vessel is made to pass between the two magnetism sensors; and
wherein the magnetism signal emitted from the inspection sample magnetized by the applied AC magnetic field is detected by the two magnetism sensors, a difference signal of outputs of the two magnetism sensors is calculated, and the difference signal is inputted to an input unit of the lock-in amplifier.

9. The magnetic field measuring apparatus according to claim 3, further comprising:
a displacement sensor of measuring a distance between the inspection sample vessel including the non-magnetic plate and the magnetism sensor;
wherein the magnetism signal emitted from the inspection sample and the distance are respectively simultaneously measured, and the magnetism signal is corrected by using a piece of distance information acquired by the displacement sensor.

10. The magnetic measuring apparatus according to claim 4, wherein in a state where a magnetism generated from the drive unit is not shielded, and when a motor of generating a magnetism noise is used, a stable magnetism measurement is realized by setting a rotational speed of the motor in a range of 1 through 10 rpm, and setting a detection band width of the lock-in amplifier in a range of 5 through 15 Hz.

11. The magnetic measuring apparatus according to claim 4,
wherein when a motor of generating a magnetism noise is used, only a main body of the motor is covered with a magnetic material having a high permeability, and
wherein a stable magnetism measurement is realized by setting a rotational speed of the motor in a range of 1 through 10 rpm, and setting a detection band width of the lock-in amplifier in a range of 5 through 15 Hz.

12. The magnetic field measuring apparatus according to claim 2, further comprising:
a non-magnetic plate mounted with the inspection sample vessel; and
a drive system configured by including a motor for linearly moving or moving to rotate the non-magnetic plate;
wherein an AC magnetic field is applied to the inspection sample by using the excitation coil while rotating the non-magnetic plate.
